(19) United States
(12) Patent Application Publication
(10) Pub. No.: US 2021/0293243 A1
(43) Pub. Date: Sep. 23, 2021

(54) PROPANE DEHYDROGENATION SYSTEM WITH SINGLE CASING REACTOR EFFLUENT COMPRESSOR AND METHOD

(71) Applicant: Nuovo Pignone Tecnologie - S.r.l., Florence (IT)

(72) Inventors: Sergio GHEZZI, Florence (IT); Dario MATINA, Florence (IT); Stefano FALOMI, Florence (IT); Giuseppe IURISCI, Barcelona (ES)

(21) Appl. No.: 17/303,933

(22) Filed: Jun. 10, 2021

Related U.S. Application Data
(63) Continuation of application No. PCT/EP2019/025457, filed on Dec. 12, 2019.

(30) Foreign Application Priority Data
Dec. 14, 2018 (IT) 102018000011099

(51) Int. Cl. F04D 17/12; C07C 5/333; B01J 38/00; F04D 29/58

(52) U.S. Cl. CPC F04D 17/122; F04D 29/5826; B01J 38/00; C07C 5/333

(57) ABSTRACT

The compression train (13) for a dehydrogenation plant (1) comprises a driver (36) and a single centrifugal compressor (35) drivingly coupled to the driver. The centrifugal compressor comprises a single casing and a plurality of compressor sections (39.1, 39.2, 39.3) inside said casing (37). Each compressor section comprises at least one impeller (40.1, 40.2) arranged for rotation in the casing (37). The compressor (35) is adapted to compress a mixture containing propane, propylene and hydrogen, having a molecular weight between 20 and 35 g/mol, from a suction pressure between about 0.2 barA and about 1.5 barA to a delivery pressure between about 11 barA and about 20 barA, with a volumetric flowrate comprised between about 120,000 m³/h and about 950,000 m³/h.

### CROSS-REFERENCE TO RELATED APPLICATIONS

This application is the U.S. national stage application filed pursuant to 35 U.S.C. 365(c) and 120 as a continuation of International Patent Application No. PCT/EP2019/025457, filed Dec. 12, 2019, which application claims priority from Italy Patent Application No. 102018000011099, filed Dec. 14, 2018, which applications are incorporated herein by reference in their entireties.

### TECHNICAL FIELD

Embodiments of the subject matter disclosed herein generally relate to dehydrogenation systems and methods. More particularly, embodiments disclosed herein concern compression trains for systems and methods for the production of propylene by propane dehydrogenation.

### BACKGROUND ART

Propylene is a colorless gaseous (at room temperature and pressure) hydrocarbon of general formula (CH₂=CH—CH₃). Propylene is used in several chemical processes, for instance for the production of polypropylene, a polymer used in a variety of applications. Propylene is presently produced as a byproduct from steam cracking of liquid feedstocks such as naphtha as well as liquefied petroleum gas (LPG) and from off-gases produced in fluid catalytic cracking units in refineries. An alternative propylene manufacturing process, to which the present disclosure relates, involves propane dehydrogenation (PDH).

Dehydrogenation of propane (CH₃CH₂CH₃) is based on the following endothermic reduction reaction:

C₃H₈ [symbol] C₃H₆+H₂ (1)

The strongly endothermic reaction is performed by contacting the propane flow with a catalyst, obtaining an effluent which is delivered from a reactor section to a product recovery section through a compression section. The compression section of the systems according to the current art includes a combination of compressors in sequence, either driven by a single driver or by multiple drivers, for instance two electric motors. The compression section has a large footprint and involves complex machinery.

Several dehydrogenation processes and plants have been developed and are known in the art, among which:
Oleflex™ dehydrogenation developed by UOP LLC, also known as Universal Oil Products LLC, USA;
CATOFIN process developed by ABB Lummus Global;
Fluidized Bed Dehydrogenation process developed by Snamprogetti, Italy;
Linde-BASF-Statoil dehydrogenation process;
Steam active reforming (STAR) technology developed by Krupp Udhe.

These processes involve a large number of machines and complex mechanical and fluid couplings. Improvements from a point of view of number of machines and footprint thereof would be beneficial.

FIG. 1 shows schematically a dehydrogenation system 101 for producing propylene according to the current art. The exemplary dehydrogenation plant 101 of FIG. 1 includes a reactor section 103, a catalyst regeneration section 105 and a product recovery section 107. The reactor section 103 includes reactors 109 arranged in sequence, i.e., in series, along a feed path 111. The feed path 111 starts from an inlet end 111A and terminates at the inlet of an effluent compression section 113.

Heater cells 115, 117.1, 117.2 and 117.3 are arranged along the feed path 111, upstream of the first reactor 109 (heater cell 115) and between each pair of sequentially arranged reactors 109 (heater cells 117.1, 117.2, 117.3). A catalyst circuit 119 delivers a catalyst flow across each reactor 109. A continuous catalyst regeneration unit 121 collects the spent catalyst from the most downstream reactor 109, regenerates and delivers the regenerated catalyst to the most upstream reactor 109.

Propane (C₃H₈) is delivered along the feed path 111 and undergoes a reduction reaction according to equation (1) above, which is promoted by heat from the heater cells 115, 117.1, 117.2, 117.3 and the catalyst. At the exit side of the feed path 111 an effluent consisting of a mixture containing propane (C₃H₈), propylene (C₃H₆) and hydrogen (H₂) is present.

The effluent at the exit side of the reactor section 103 has a low pressure value, typically below ambient pressure, and must be pressurized at a high pressure for recovering the components thereof in the product recovery section 107. The compression section 113 provides for compression of the effluent and delivery of the compressed effluent through the product recovery section 107. The product recovery section 107 includes a drier 131 and a liquid/gas separator 133, where hydrogen and propane can be separated from propylene, which is collected at the bottom of the separator 133 and further processed, e.g., polymerized to produce polypropylene.

The recovered hydrogen and propane are expanded in a turbo-expander 134 and re-cycled towards the inlet end 111A of the feed path 111.

The compression section 113 comprises a compression train 141 including a plurality of separate compressors arranged in series. In the schematic of FIG. 1 the compression train 141 comprises a first compressor 143 and a second compressor 145 arranged in two separate compressor casings and drivingly coupled to a shaftline 147, A driver 149, for instance an electric motor or a turbine, drives the compressors 143, 145 into rotation.

The compression train 141, including at least three rotary machines and a relevant shaftline connecting the plurality of compressors to the driver, is a critical part of the plant 101 and involves a large footprint. The large number of machines and machine components of the compression train renders the compression section expensive to install and run, energy consuming and prone to failures. Costly and frequent maintenance interventions are required.

A need therefore exists to improve the dehydrogenation plants for propylene production, aiming at overcoming or alleviating the drawbacks of the current art plants.

### SUMMARY

According to a first aspect of the present disclosure, a compression train for a dehydrogenation plant for propylene production is provided. The compression train includes a driver and a single centrifugal compressor drivingly coupled to the driver. The driver can be any source of mechanical power adapted to rotate the compressor.

According to embodiments disclosed herein, the centrifugal compressor includes a single casing and a plurality of compressor sections inside said casing. Each compressor section includes at least one impeller arranged for rotation in the casing.

The compressor is configured to compress a mixture containing propane, propylene and hydrogen, having a molecular weight between about 20 and about 35 g/mol, from a suction pressure between about 0.2 barA and about 1.5 barA to a delivery pressure between about 11 barA and about 20 barA, with a volumetric flowrate comprised between about 120,000 m³/h and about 950,000 m³/h.

According to a further aspect, disclosed herein is a plant for the production of propylene by propane dehydrogenation. The plant comprises a reactor section, a catalyst regeneration section, a product recovery section and a compression train between the reactor section and the production recovery section. The compression train is adapted to pressurize and feed a flow of effluent from the reactor section to the product recovery section. The compression train can include a driver and single centrifugal compressor as defined above.

According to yet another aspect, disclosed herein is a method for producing propylene by dehydrogenation of propane in a dehydrogenation plant. A first step of the method comprises conducting a catalytic reduction reaction of propane in a reactor section of said dehydrogenation plant. Effluent containing propylene is collected from the reaction section and is compressed from a first, low pressure at an exit side of the reactor section, to a second, high pressure at an inlet of a product recovery section of the dehydrogenation plant. Compression of the effluent is performed using a single compressor having a single casing and a plurality of compressor sections inside said casing, each compressor section comprising at least one impeller arranged for rotation in the casing, said single compressor adapted to compress the effluent from a first, low pressure at the outlet of the reactor section to a second, high pressure at the inlet of the product recovery section.

Further advantageous features and embodiments of the method and system of the present disclosure are described below and are set forth in the attached claims.

### BRIEF DESCRIPTION OF THE DRAWINGS

A more complete appreciation of the disclosed embodiments of the invention and many of the attendant advantages thereof will be readily obtained as the same becomes better understood by reference to the following detailed description when considered in connection with the accompanying drawings, wherein:

FIG. 1 illustrates a schematic of a propane dehydrogenation plant according to the current art;

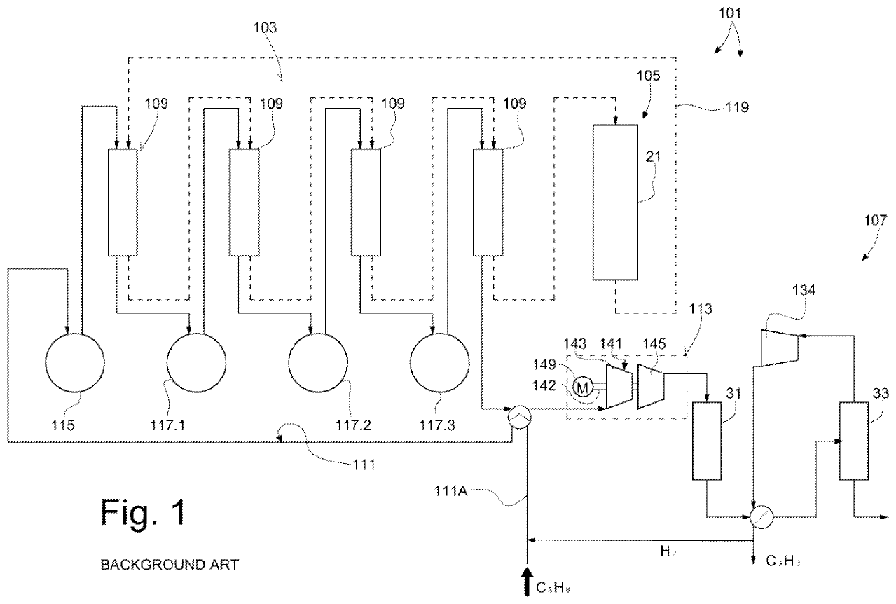

FIG. 2 illustrates a schematic of a propane dehydrogenation plant according to the present disclosure;

FIGS. 3, 4 and 5 illustrate three configurations of a two-sections high pressure ratio compressor for the system of FIG. 2;

FIGS. 6, 7, 8, 9 and 10 illustrate five configurations of a three-sections high pressure ratio compressor for the system of FIG. 2; and

FIG. 11 illustrate a flow chart summarizing a method according to the present disclosure.

### DETAILED DESCRIPTION

A new and useful compression train for a plant for the production of propylene through propane dehydrogenation has been developed. As mentioned above, propylene is an aliphatic hydrocarbon of general formula CH₂=CH—CH₃ obtained by dehydrogenation of propane, i.e., by removing one hydrogen atom from the propane molecule (CH₃CH₂CH₃) and obtaining hydrogen (H₂) and propylene. One part of the process involves compressing a gaseous mixture of propane, hydrogen and propylene, delivered by a reactor section of the dehydrogenation plant at low pressure, usually below ambient pressure, and temperatures ranging between about 30 and about 70° C. The propylene, hydrogen and propane gaseous mixture, usually referred to as effluent, shall be compressed at high pressure values, up to about 11 barA and above, for instance up to about 15 barA and above.

In the past, the effluent was compressed using large, multi-casing compression trains, including at least two compressor casings separate from one another and drivingly coupled to a shaft line, driven into rotation by a driver. These compression trains took up lots of space. Now, it has been discovered that the compression train can be made smaller by using a single compressor, with a single casing housing a plurality of compressor sections. In so doing the footprint (and foundation works) of the compression train can be reduced. In some embodiments up to 50% reduction in the footprint of the compression train can be achieved.

The total power consumption for driving the compression train of the present disclosure is the same or can be lower than the power required to drive a compression train of the current art.

The full pressure increase from the effluent low pressure at the exit of the reactor section of the dehydrogenation plant, to the effluent high pressure at the inlet side of the product recovery section is obtained through a single, multi-stage, centrifugal compressor. In particularly advantageous embodiments, the compressor is a high pressure ratio compressor (HPRC). Besides an overall footprint and foundation works reduction, using a compression train having a single compressor casing also reduces the number of ancillary devices and machinery, such as seals, drivers and gear boxes, thus increasing reliability and availability of compression train.

As understood herein the casing of a compressor is the component thereof which houses the compressor rotor and which extends from a suction side, where process fluid at the low, suction pressure enters the compressor, to a delivery side, where process fluid at the high, delivery pressure exits the compressor. In a propane dehydrogenation plant for the production of propylene the suction pressure is the pressure at which the effluent exits the reactor section and the delivery pressure is the pressure at which the effluent enters the product recovery section.

Differently from the systems of the prior art, the compression train and relevant method disclosed herein perform the entire pressure increase from the reactor section to the product recovery section of the propane dehydrogenation plant in a single casing compressor. The full compression step is performed in the single casing. No further compressors are required downstream of the delivery side of the single compressor.

As will be described here below, the efficiency of the compression train can be improved by providing intercooling between at least two sections of the compressor.

The single compressor of the compression train can be a vertically split compressor. As used herein, the term “vertically split” indicates a compressor, the casing whereof can be opened along a vertical plane. In some embodiments, the casing can comprise a central barrel and one removable terminal closure, or two opposite terminal closures at two axially opposed ends of the casing. In other embodiments, the single compressor can be a horizontally split compressor. As used herein, the term “horizontally split” indicates a compressor, the casing whereof is comprised of two portions coupled to one another along a horizontal plane and which can be separated to open the compressor casing.

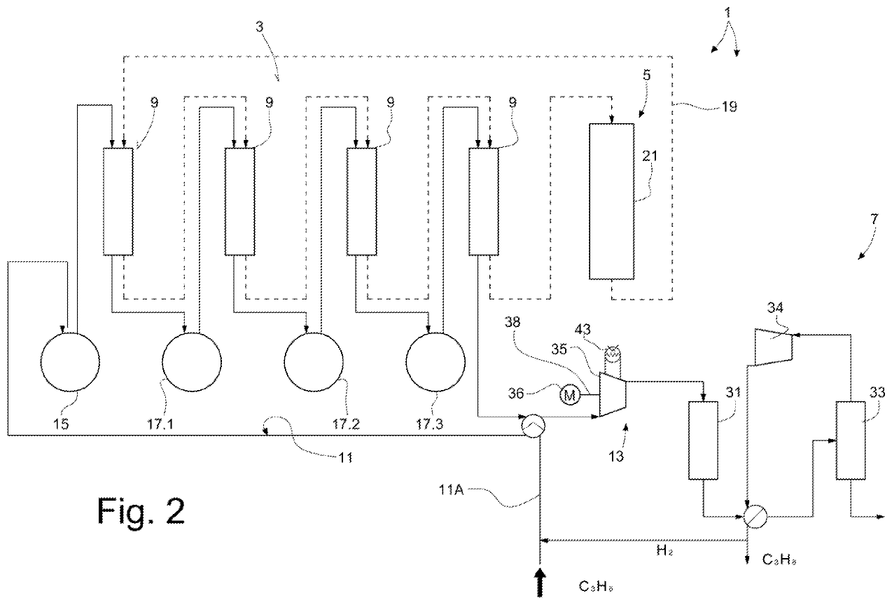

FIG. 2 shows a dehydrogenation plant 1 for producing propylene. The general structure of the plant is known and can vary depending upon the technology used. In general terms, the novel compression train of the present disclosure can be used in any dehydrogenation plant for polypropylene production, wherein an effluent, comprised of a mixture of propane, propylene and hydrogen, must be recovered at a low pressure exit side of a reaction section of the plant and compressed to a higher pressure at the inlet of a product recovery section. Therefore, the novel features of the compression train disclosed herein can be implemented in dehydrogenation plants differing from the one shown in FIG. 2.

The exemplary dehydrogenation plant 1 of FIG. 2 includes a reactor section 3, a catalyst regeneration section 5 and a product recovery section 7. The reactor section 3 includes one or more reactors 9, which are arranged in sequence along a feed path 11, which extends from an inlet end 11A and terminates at a suction side of an effluent compression train 13.

Heater cells 15, 17.1, 17.2 and 17.3 are arranged along the feed path 11, upstream of the first reactor 9 and between each pair of sequentially arranged reactors 9, A catalyst circuit 19 delivers a catalyst flow across each reactor 9. A continuous catalyst regeneration unit 21 collects the spent catalyst from the most downstream reactor 9, regenerates and delivers the regenerated catalyst to the most upstream reactor 9.

Propane (C₃H₈) is delivered along the teed path 11 and undergoes a reduction reaction promoted by heat from the heater cells 15, 17.1, 17.2, 17.3 and the catalyst. At the exit side of the feed path 11 an effluent consisting of a gaseous mixture containing propane (C₃H₈), propylene (C₃H₆) and hydrogen (H₂) is present. Examples of effluent compositions and other operating parameters will be given later on.

The compression train 13 boosts the pressure of the effluent and delivers the compressed effluent to the product recovery section 7. In some embodiments, as shown by way of example in FIG. 2, the product recovery section 7 can include a drier 31 and a liquid/gas separator 33, where hydrogen and propane can be separated from propylene, which is collected at the bottom of the separator 33 and further processed, e.g., polymerized to produce polypropylene.

The recovered hydrogen and propane can be expanded in a turbo-expander 34, for instance, for energy recovery purposes and re-cycled towards the inlet end 11A of the feed path 11 and/or to the gas separator 33.

The pressurization from the low pressure at the exit side of the reactor section 3 (here on referred to also as “first pressure”) to the high pressure at the inlet side of the product recovery section 7 (here on referred to also as “second pressure”) is performed by the compression train 13, which includes a single centrifugal compressor, and specifically a single, high pressure ratio compressor, for instance.

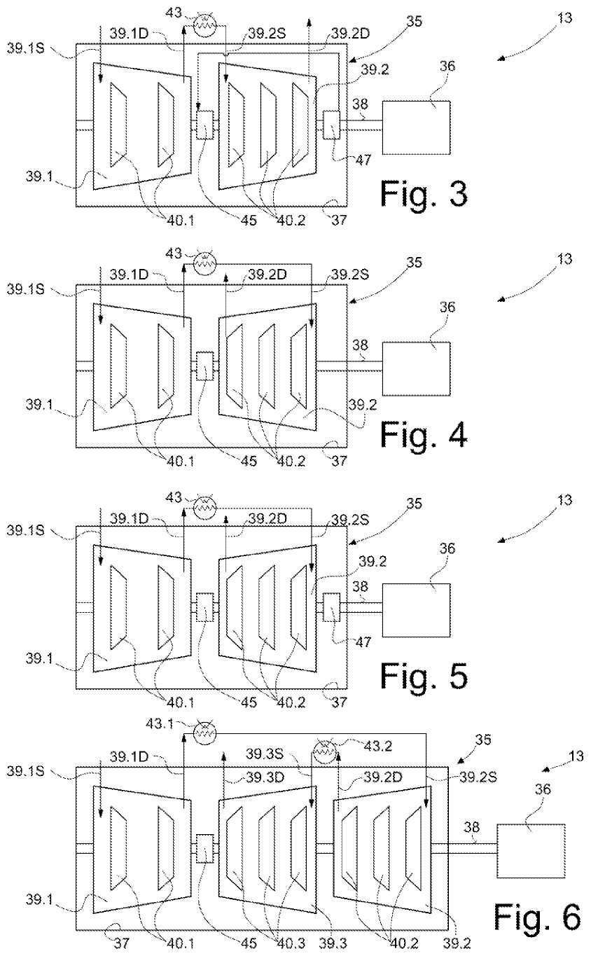

FIG. 3 shows a first embodiment of the compression train 13, which can be used in the dehydrogenation plant 1 of FIG. 2 and which includes a single centrifugal compressor. The compressor is labeled 35 and can be driven into rotation by a driver 36 through a shaft line 38. The driver can be an electric motor, or a steam turbine, for instance. In other embodiments, a gas turbine engine can be used as prime mover, i.e. as a driver for the compressor 35. The driver can be connected to the compressor with or without a gearbox therebetween.

The compressor 35 comprises s single casing 37, wherein a plurality of compressor stages can be arranged. Each compressor stage can comprise a centrifugal impeller arranged for rotation in the compressor casing 37. In other embodiments, a compressor stage can include a plurality of compressor impellers. The centrifugal compressor stages can be grouped in a plurality of centrifugal compressor sections, for instance two or three centrifugal compressor sections.

Each centrifugal impeller can be a shrouded impeller or an unshrouded impeller. In some embodiments, the compressor 35 can comprise a combination of shrouded impellers and unshrouded impellers. For instance, a centrifugal compressor section can include only shrouded impellers and another centrifugal compressor section can include only unshrouded impellers. In other embodiments at least one, some or all centrifugal compressor sections can include a combination of shrouded impellers and unshrouded impellers.

The compressor 35 can include one or more centrifugal compressor sections, each including at least one stacked impeller or a plurality of sequentially arranged stacked impellers. If only one axially stacked impeller is provided, the impeller is axially stacked with two portions of an axial shaft.

Axially stacked impellers allow high rotational speeds of the compressor rotor and are therefore particularly useful in the range of pressure ratios involved in the configurations disclosed herein. As usually understood in the art, axially stacked impellers are impellers, which are stacked one on the other along a rotation axis and are mutually coupled to one another in order to transfer a torque from one impeller to the other, or from an impeller to a shaft portion, by means of a Hirth coupling or similar connections. As known to those skilled in the art, a Hirth coupling, also referred to as Hirth joint, uses tapered teeth on opposing ends of two shafts to be coupled to one another. The tapered teeth mesh together to transmit torque from one shaft to the other.

In some embodiments, the compressor 35 can include one or more radial shrink fit impellers. As known to those skilled in the art of centrifugal compressors, shrink-fit impellers are mounted on a central shaft which connect the impellers to one another.

In some embodiments, the compressor 35 can include a combination of radial shrink fit impellers and axially stacked impellers.

In the exemplary embodiment of FIG. 3 two centrifugal compressor sections 39.1 and 39.2 are arranged in the casing 37. Each centrifugal compressor section 39.1 and 39.2 can includes a plurality of centrifugal compressor impellers schematically shown at 40.1 (for section 39.1) and 40.2 (for section 39.2). In the embodiment of FIG. 3 the centrifugal compressor sections 39.1 and 39.2 are arranged according to an in-line configuration. As used herein the term “in-line” indicates a configuration in which the gas flows in the two sections globally in the same direction. In FIG. 3 the effluent gas flows through the first section 39.1 and through the second section 39.2 from the left to the right. The numbering of the centrifugal compressor sections (“first” and “second” centrifugal compressor section) in FIG. 3 as well as in the subsequent FIGS. 4 to 10 is according to the pressure increase through the compressor 35, i.e., the first centrifugal compressor section 39.1 is the one at lower pressure and is arranged upstream of the second centrifugal compressor section 39.2, such that the effluent is compressed sequentially in the first centrifugal compressor section and 39.1 and subsequently in the second centrifugal compressor section 39.2.

In order to improve the efficiency of the compressor, in some embodiments the effluent flow is cooled in an intercooler fluidly coupled between the first com centrifugal pressor section 39.1 and the second centrifugal compressor section 39.2.

More specifically, the first centrifugal compressor section 39.1 comprises a suction side 39.1S and a delivery side 39.1D. The effluent enters the first centrifugal compressor section 39.1 at the suction side 39.1S and exits the first centrifugal compressor section 39.1 at the delivery side 39.1D and sequentially enters the second centrifugal compressor section 39.2 at a suction side 39.2S and exits from the second centrifugal compressor section 39.2 at a respective delivery side 39.2D. Between the delivery side 39.1D and the suction side 39.2S the effluent is cooled in an intercooler 43.

In some embodiments, the compressor 35 can comprise a first balance drum 45 between the first centrifugal compressor section 39.1 and the second centrifugal compressor section 39.2. The compressor can include a second balance drum 47 arranged at the delivery side of the second centrifugal compressor section 39.2. Alternatively, the balance drum 47 can be arranged at suction side of the first centrifugal compressor section 39.1.

In some embodiments, the temperature at the suction side of the compressor 35 can be comprised between about 35° C. and about 65° C.

Unless differently specified, as used herein the term “about” when referred to a value of a parameter or quantity, can be understood as including any value within ±5% of the stated value. Thus, for instance, a value of “about x”, includes any value within the range of (x−0.05x) and (x+0.05x).

In some embodiments, the low pressure at the exit of the reactor section 3 can be comprised between about 0.5 barA (bar absolute) and about 1.1 barA, preferably around 0.8 barA. The delivery pressure of the compressor 35 can be comprised between about 13 barA and about 19 barA, preferably between about 14 barA and about 16 barA, more preferably around 15 barA. The compressor 35 can have a volumetric flowrate comprised for instance between about 120,000 and about 600,000 m³/h, preferably between about 150,000 and about 500,000 m³/h. As commonly understood in the art, the volumetric flowrate is the flowrate at the suction side of the compressor.

The effluent can comprise a mixture as follows, expressed in % MOL:

| | |
|---|---|
| Propane | 30-34% |
| Propylene | 13-17% |
| Hydrogen | 44-49% |

with a molecular weight ranging around 23-24 g/mol, in particular about 23.4 g/mol.

According to other embodiments, the low pressure at the exit of the reactor section 3 can be comprised between about 0.2 barA and about 0.4 barA, preferably around 0.3 barA. The delivery pressure of the compressor 35 can be comprised between about 11 barA and about 15 barA, preferably between about 12 barA and about 14 barA, more preferably around 13 barA. The compressor can have a volumetric flowrate comprised for instance between about 120,000 and about 850,000 m³/h, preferably between about 150,000 and about 750,000 m³/h at the suction side of the compressor 35.

The effluent can comprise a mixture as follows, expressed in % MOL:

| | |
|---|---|
| Propane | 33-36% |
| Propylene | 23-25% |
| Hydrogen | 29-31% |

with an average molecular weight of about 29 g/mol.

While in FIG. 3 a compressor 35 in an in-line configuration is shown, other compressor configurations are possible, such as a back-to-back configuration. FIGS. 4 and 5 illustrate schematically two embodiments of a high pressure ratio compressor 35 in a back-to-back configuration. The same reference numbers used in FIG. 3 are used in FIGS. 4 and 5 to designate the same or corresponding parts, which are not described again. As used herein the term “back-to-back” is understood as a configuration in which the effluent flows in opposite directions in the two compressor sections. For instance, in FIG. 4 the effluent flows from the left to the right in the first centrifugal compressor section 39.1 and from the right to the left in the second centrifugal compressor section 39.2.

The compressors of FIGS. 4 and 5 differ from one another mainly in view of the balance drum arrangement. While in FIG. 4 only a balance drum 45 arranged between the two centrifugal compressor sections 39.1 and 39.2 is provided, in FIG. 5 a second balance drum 47 is provided at the suction side of the second centrifugal compressor section 39.2. Alternatively, the balance drum 47 can be arranged at suction side of the first centrifugal compressor section 39.1.

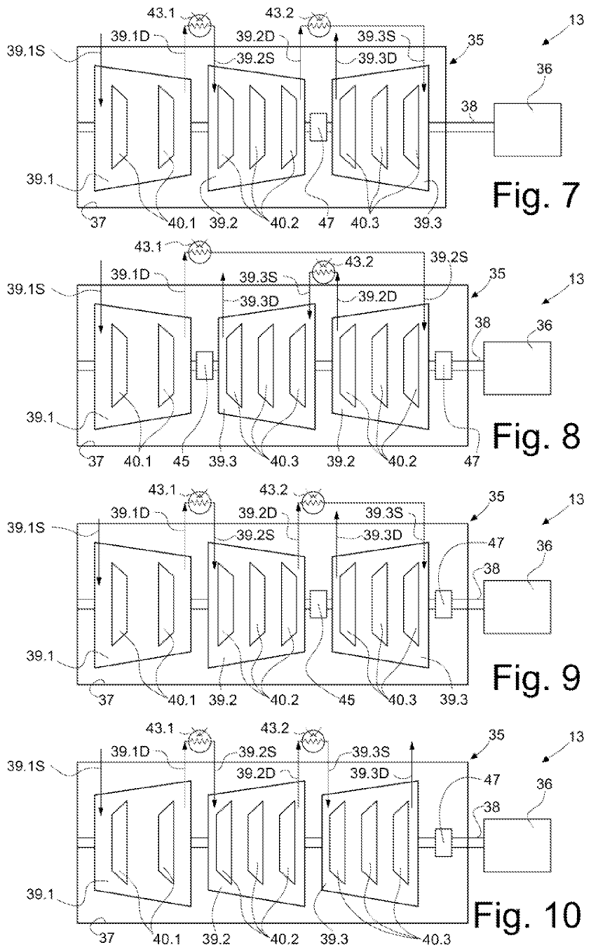

In some embodiments, the compressor 35 may comprise more than two centrifugal compressor sections. FIGS. 6, 7, 8, 9 and 10 illustrate five embodiments of compressors 35, each including three centrifugal compressor sections, labeled 39.1, 39.2 and 39.3, respectively. For instance, the compressor 35 of FIG. 6 comprises a single casing 37 containing three centrifugal compressor sections 39.1, 39.2 and 39.3. In the exemplary embodiment of FIG. 6 the first and second centrifugal compressor sections 39.1 and 39.2 are arranged on opposite sides of the third centrifugal compressor section 39.3, which is located centrally. In the present disclosure, unless differently indicated, the sections are sequentially numbered according to the increasing pressure, i.e., the process gas pressure increases when moving from the first centrifugal compressor section 39.1 to the second centrifugal compressor section 39.2 and from this latter to the third centrifugal compressor section 39.3. A balance drum 45 is arranged between the first centrifugal compressor section 39.1 and the third centrifugal compressor section 39.3.

Each centrifugal compressor section includes a suction side, designated with the reference number of the centrifugal compressor section followed by the letter S, as well as a delivery side, labeled with the reference number of the centrifugal compressor section, followed by the letter D. The delivery side 39.1D of the first centrifugal compressor section 39.1 is fluidly coupled to the suction side 39.2S of the second centrifugal compressor section 39.2 through a first intercooler 43.1. Similarly, the delivery side 39.2D of the second centrifugal compressor section 39.2 is fluidly coupled to the suction side 39.35 of the third centrifugal compressor section 39.3 through a second intercooler 43.2.

In other embodiments only one intercooler can be provided, for instance only intercooler 43.1 or only intercooler 43.2.

In the embodiment of FIG. 6 the first centrifugal compressor section 39.1 and the third centrifugal compressor section 39.3 are arranged in a back-to-back configuration, while the second centrifugal compressor section 39.2 and the third centrifugal compressor section 39.3 are arranged in an in-line configuration.

FIG. 7 illustrates a further high pressure ratio compressor 35 with three centrifugal compressor sections 39.1, 39.2, 39.3. The compressor of FIG. 7 differs from the compressor of FIG. 6 mainly in view of the different position of the balance drum and of the sequence of first, second and third centrifugal compressor sections. The balance drum 45 is located between the second centrifugal compressor section 39.2 and the third centrifugal compressor section 39.3. Moreover, the first centrifugal compressor section 39.1 and the second centrifugal compressor section 39.2 are in an in-line configuration, while the second centrifugal compressor section 39.2 and the third centrifugal compressor section 39.3 are arranged in a back-to-back configuration.

A further embodiment of a compressor 35 for use in the dehydrogenation plant 1 of FIG. 2 is shown in FIG. 8. The same reference numbers of FIGS. 6 and 7 designate the same or corresponding parts, which are not described again. The compressor 35 of FIG. 8 differs from the compressor 35 of FIG. 6 mainly in view of a second balance drum 47 arranged on the suction side of the second centrifugal compressor section 39.2. Alternatively, balance drum 47 can be arranged at suction side of the first centrifugal compressor section 39.1.

FIG. 9 illustrates a yet further embodiment of a high pressure ratio compressor 35 which differs from the compressor of FIG. 7 in view of an additional balance drum 47 arranged on the suction side of the third centrifugal compressor section 39.3. Alternatively, the additional balance drum 47 can be arranged at the suction side of the first centrifugal compressor section 39.1.

While FIGS. 6, 7, 8 and 9 illustrate embodiments wherein two adjacent centrifugal compressor sections are in a back-to-back configuration, FIG. 10 illustrates a further embodiment, wherein three centrifugal compressor sections 39.1, 39.2 and 39.3 are arranged in an in-line configuration. A single balance drum 37 is positioned on the suction side of the third centrifugal compressor section 39.3.

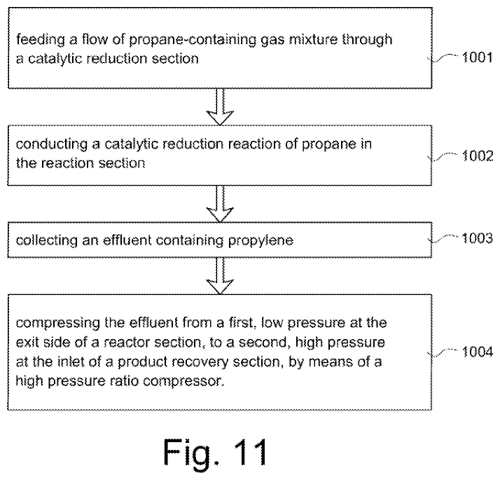

With reference to FIG. 11, an operating cycle of the dehydrogenation plant 1 using the new and useful compression train is now described. Reference 1001 indicates a step of feeding a flow of propane-containing gas mixture through the catalytic reduction section. The step 1002 involves conducting a catalytic reduction reaction of propane in the reactor section. The cycle further includes (step 1003) collecting an effluent containing propylene from the reaction section. The effluent is compressed (step 1004) from a first, low pressure at the exit side of the reactor section, to a second, high pressure at an inlet of the product recovery section of the dehydrogenation plant 1, using a single compressor 35.

While the invention has been described in terms of various specific embodiments, it will be apparent to those of ordinary skill in the art that many modifications, changes, and omissions are possible without departing form the spirt and scope of the claims. In addition, unless specified otherwise herein, the order or sequence of any process or method steps may be varied or re-sequenced according to alternative embodiments.

1. A compression train for a dehydrogenation plant, comprising:
a driver;
a single centrifugal compressor drivingly coupled to the driver;
wherein the centrifugal compressor comprises a single casing and a plurality of centrifugal compressor sections inside said casing, each centrifugal compressor section comprising at least one impeller arranged for rotation in the casing, said compressor adapted to compress a mixture containing propane, propylene and hydrogen, having a molecular weight between about 20 and about 35 g/mol, from a suction pressure between about 0.2 barA and about 1.5 barA to a delivery pressure between about 11 barA and about 20 barA, with a volumetric flowrate comprised between about 120,000 m³/h and about 950,000 m³/h.

2. The compression train of claim 1, wherein at least one of said centrifugal compressor sections comprises a plurality of impellers.

3. The compression train of claim 1 wherein at least one of said centrifugal compressor sections includes at least one axially stacked impeller.

4. The compression train of claim 1 wherein at least one of said impellers is an unshrouded impeller.

5. The compression train of claim 1 wherein at least two of said centrifugal compressor sections are arranged in an in-line configuration.

6. The compression train of claim 1 wherein at least two of said centrifugal compressor sections are arranged in a back-to-back configuration.

7. The compression train of claim 1, including an intercooler between at least two of said centrifugal compressor sections.

8. The compression train of claim 1, wherein the suction pressure is comprised between about 0.2 barA and about 1.1 barA.

9. The compression train of claim 1, wherein the delivery pressure is comprised between about 11 barA and about 19 barA.

10. The compression train of claim 1, wherein the suction pressure is comprised between about 0.5 and about 1.1 barA and the delivery pressure is comprised between about 13 barA and about 19 barA.

11. The compression train of claim 1, wherein the suction pressure is comprised between about 0.2 and about 0.4 barA and the delivery pressure is comprised between about 11 barA and about 15 barA.

12. The compression train of claim 1, wherein the volumetric flowrate is comprised between about 150,000 m³/h and about 750,000 m³/h.

13. The compression train of claim 1, wherein the gas mixture at the suction side of the compressor has a temperature comprised between about 30° C. and about 70° C.

14. The compressor train of claim 1, wherein said single centrifugal compressor comprises at least a first compressor section including at least one unshrouded and axially stacked impeller and a second compressor section including at least one shrouded and radial shrink-fit impeller.

15. A system for the production of propylene by propane dehydrogenation, comprising:
a reactor section;
a catalyst regeneration section;
a product recovery section; and
between the reactor section and the production recovery section, a compression train according to claim 1, adapted to feed a flow of effluent from the reactor section to the product recovery section.

16. A method for producing propylene by dehydrogenation of propane in a dehydrogenation plant, the method comprising the steps of:
conducting a catalytic reduction reaction of propane in a reactor section of said dehydrogenation plant;
collecting an effluent containing propylene from the reactor section; and
compressing the effluent from a first, low pressure at an exit side of the reactor section, to a second, high pressure at an inlet of a product recovery section of said dehydrogenation plant using a single compressor having a single casing and a plurality of compressor sections inside said casing, each section comprising at least one impeller arranged for rotation in the casing, said single compressor adapted to compress the effluent from a first, low pressure at the outlet of the reactor section, comprised between about 0.2 barA and about 1.5 barA, to a second, high pressure at the inlet of the product recovery section, comprised between about 11 barA and about 20 barA; wherein the compressor has a volumetric flowrate comprised between about 120,000 m³/h and about 950,000 m³/h.

17. The method of claim 16, comprising the step of intercooling the effluent between at least two sequentially arranged compressor sections.

18. The method of claim 16, wherein the second, high pressure is comprised between about 11 barA and about 19 barA.

19. The method of claim 16, wherein the first, low pressure is comprised between about 0.5 and about 1.1 barA and the second, high pressure is comprised between about 13 barA and about 19 barA.

20. The method of claim 16, wherein the first, low pressure is comprised between about 0.2 barA and about 0.4 barA, and the second, high pressure is comprised between about 11 barA and about 15 barA.

21. The method of claim 16, wherein the compressor has a volumetric flowrate comprised between about 150,000 m³/h and about 750,000 m³/h.

22. The method of claim 16, wherein the effluent at the suction side of the compressor has a temperature comprised between about 30° C. and about 70° C.
